(19) United States
(12) Patent Application Publication
(10) Pub. No.: US 2005/0260762 A1
(43) Pub. Date: Nov. 24, 2005

(54) Method of in vitro evaluation of the retention and/or the transfer properties of cosmetics

(75) Inventor: Franck Giron, Ferrieres En Brie (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 10/946,315

(22) Filed: Sep. 22, 2004

(30) Foreign Application Priority Data
Sep. 26, 2003 (FR) 03 11287

(51) Int. Cl. G01N 21/75; A61K 7/06; A61K 7/11

(52) U.S. Cl. 436/164

(57) ABSTRACT

The present invention relates to a method of in vitro evaluation of the transfer of a cosmetic comprising: raising a first medium to a predetermined temperature; depositing a film of the cosmetic on a first face of the first medium; allowing the film to dry during a predetermined duration; measuring the color of a second medium and the color of a total transfer reference; forming a first imprint of the film of cosmetic on the second medium by pressing the first face of the first medium in static manner against the second medium with predetermined pressure; measuring the color of the first imprint; and evaluating the transfer of the cosmetic from the colors as measured in this way.

The present invention relates to in vitro evaluation of the transfer and/or the retention properties of a cosmetic, particularly but not exclusively of a lipstick.

### BACKGROUND OF THE INVENTION

In order to test the transfer or the retention of a lipstick, it is common practice to perform evaluations in vivo.

For example, in order to evaluate the transfer of a lipstick, it can be applied to the lips of a person, and the person can be asked to press the lips against a piece of paper. The imprint that this forms is compared with images in an atlas in order to evaluate transfer.

Another in vivo test for evaluating the retention of lipstick consists in causing a person to take a particular meal after applying the lipstick to the lips. At the end of the meal, the residual color of the lips is evaluated.

These in vivo tests are awkward to implement. In addition, a toxicological permit must be obtained prior to applying any new composition, and that further complicates performing the test.

There exists a need to benefit from an in vitro test that makes it possible to evaluate the transfer and/or the retention of a cosmetic, and that is capable of replacing the above-mentioned in vivo test.

U.S. Pat. No. 6,340,466 discloses a test for in vitro evaluation of the non-transfer properties of a cosmetic. That test makes use in particular of a medium constituted by a sausage casing for rehydrating in a predetermined manner, and onto which a film of the cosmetic is applied. After the film has been transferred onto a medium by applying rotation and pressure, the reflectance of the film that has been transferred onto said medium is measured. That test which is relatively complicated to perform, is not entirely satisfactory.

### SUMMARY OF THE INVENTION

In a first of its aspects, the present invention provides a method for in vitro evaluation of the transfer of a cosmetic, for example a lipstick, said method comprising:
raising a first medium to a predetermined temperature;
depositing a film of the cosmetic on a first face of the first medium;
allowing the film to dry during a predetermined duration;
measuring the color of a second medium and the color of a total transfer reference;
forming a first imprint of the film of cosmetic on the second medium by pressing the first face of the first medium in static manner against the second medium with predetermined pressure;
measuring the color of the first imprint; and
evaluating the transfer of the cosmetic from the colors as measured in this way, for example from the ratio of the color difference between the first imprint and the second medium having no imprint, relative to the color difference between the total transfer reference and the second medium having no imprint.

In another aspect the present invention also provides a method of in vitro evaluation of the retention of a cosmetic, for example a lipstick, the method comprising:
raising a first medium to a predetermined temperature;
measuring the color of a first face of the first medium;
depositing a film of the cosmetic on the first face of the first medium;
allowing the film of the cosmetic to dry during a first predetermined duration;
measuring the color of the film of cosmetic;
forming a first imprint of the film of cosmetic on a second medium by pressing the first face of the first medium in static manner against the second medium with predetermined pressure;
depositing at least one predetermined quantity of at least one liquid on the second medium at at least one predetermined location;
forming a second imprint of the film of cosmetic on the second medium by pressing the first face of the first medium against the second medium with predetermined pressure and by moving the second medium relative to the first face of the first medium at a predetermined speed and along a predetermined path passing through the deposit(s) of liquid;
measuring the residual color of the film of cosmetic on the first medium; and
evaluating the retention of the cosmetic on the basis of the colors as measured in this way, for example from the ratio of the color difference between the film after the two imprints and the first face of the first medium having no film, relative to the color difference between the film before the first two imprints and the first face of the first medium having no film.

The results obtained by such methods are similar to those that can be obtained using an in vivo method of evaluation.

### BRIEF DESCRIPTION OF THE DRAWINGS

The present invention can be better understood on reading the following detailed description of non-limiting embodiments of the invention and on examining the accompanying drawings, in which:

FIG. 1 is a diagrammatic side view of the first medium used for the non-transfer test;

FIG. 2 is a diagrammatic plan view of the first medium, on which a film of cosmetic has been deposited;

FIG. 3 is a diagrammatic axial section of the first medium of FIG. 2 once it has been fixed on a sample carrier;

FIG. 4 is a diagrammatic plan view of a sample cut out from the first medium of FIGS. 2 and 3;

FIG. 5 is a diagrammatic perspective view of the sample carrier and the sample of FIG. 4;

FIG. 6 shows the second medium before the transfer test;

FIG. 7 shows the second medium after the transfer test;

FIG. 8 is a diagrammatic side view of the first medium used for the retention test;

FIG. 9 is a diagrammatic plan view of the first medium on which a cosmetic film has been deposited;

FIG. 10 is a diagrammatic axial section of the first medium of FIG. 9 after it has been fixed on a sample carrier;

FIG. 11 is a diagrammatic plan view showing a sample cut out from the first medium of FIGS. 9 and 10;

FIG. 12 is a diagrammatic perspective view of the sample carrier and the sample of FIG. 11;

FIG. 13 shows the second medium before the retention test; and

FIG. 14 shows the second medium after the retention test.

### DETAILED DESCRIPTION OF EXEMPLARY EMBODIMENTS

Non-transfer test FIG. 1 shows a first medium 1 of rectangular shape having dimensions of 40 millimeters (mm)×70 mm and comprising a layer 2 of foam, having a white color, e.g. a polyethylene foam with density of 33 kilograms per cubic meter (kg/m³), and thickness of 3 mm, and in particular a foam known under the trade reference RE40X70 EP3 and sold by the supplier Joint Technique Lyonnais Ind.

The first medium 1 has an adhesive surface 4 on one of its faces.

A protective film 5 covers this adhesive surface 4. The protective film 5 includes a pull tab 5a to make it easier to remove.

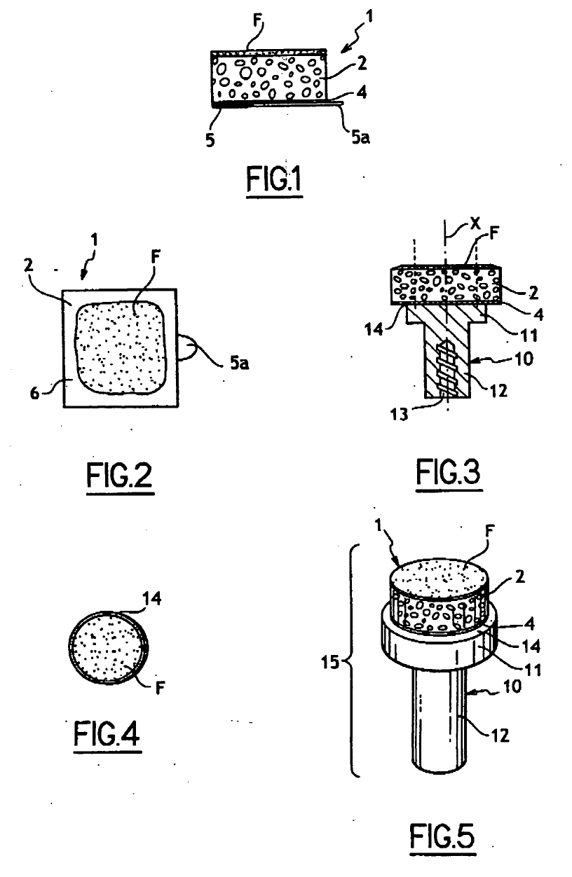

An implementation of the method of evaluating transfer in accordance with the invention is described below with reference to FIGS. 2 to 7.

The operator places the first medium 1 on a hot-plate set to the temperature of 40° C., e.g. a hot-plate of the N 81076 type sold by the supplier Ficher Bioblock, and waits until the first face 6 of the first medium 1 reaches a temperature of 33° C.±1° C., which “first” face is defined as the face remote from that carrying the adhesive surface 4. In order to monitor the surface temperature of the first medium 1, the operator can use an infrared thermometer.

Once the first medium 1 is at the desired temperature, the operator applies a film F of a cosmetic to a thickness of about 15 micrometers (μm) on the first face 6 of the first medium 1.

The cosmetic, which is constituted for example by a lipstick, is previously stored at 24° C.±2° C.

The operation performed by the operator to deposit the film of cosmetic may consist, for example, in making zigzags on the first medium so as to cover substantially all of its surface, while generally passing only once over any one location. The cosmetic is preferably applied to the first medium in a manner that is as representative as possible of real conditions in which the cosmetic is applied.

The operator can then smooth the deposited film using a paint brush.

Thereafter, the film is allowed to dry, the first medium 1 remaining on the hot-plate so that it remains at 33° C.±1° C. for a duration of 30 minutes (min) in the example described.

The operator then proceeds to fix, in particular by means of adhesive, the first medium 1 on a sample carrier 10 made of aluminum as shown in FIG. 3.

To do this, the operator removes the protective film from the first medium 1 and fixes said medium via its adhesive surface 4 on the sample carrier 10, which sample carrier has a head 11 and a body 12 provided with a tapped hole 13.

The first medium 1 is stuck on the substantially circular and plane top face 14 of the head 11, e.g. having a diameter equal to 20 mm, using a zone of the medium in which the film F of cosmetic is uniform.

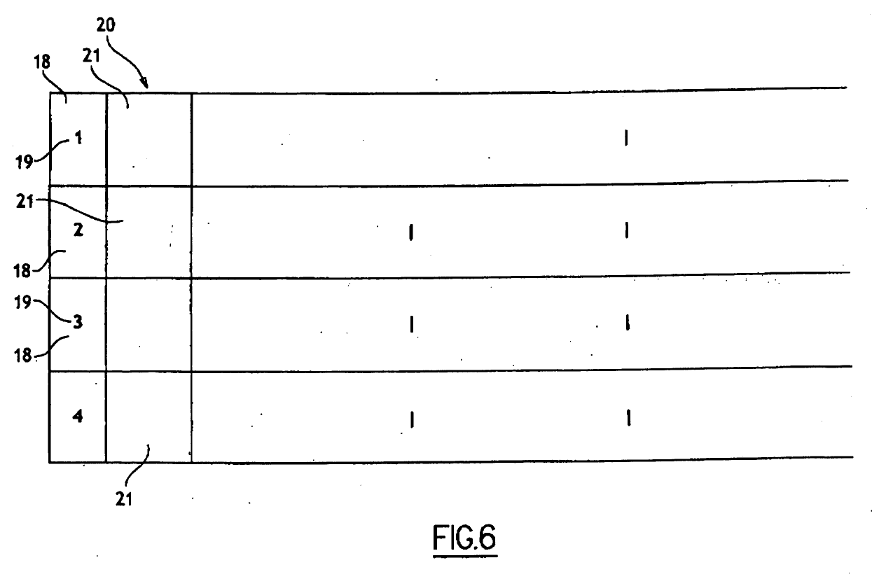

By using a punch, the operator cuts out from the first medium 1 a circle of diameter that is slightly smaller than that of the head 11, e.g. equal to 18 mm, as can be seen in FIG. 4.

The assembly comprising the sample carrier and the sample constitutes a test piece 15 as shown in FIG. 5.

The operator can prepare a plurality, in particular a total of four, test pieces 15 that are identical and as shown in FIG. 5.

Thereafter, the operator mounts each sample carrier 12 on a dynamometer press, using the tapped hole 13 for screwing it onto the press.

By way of example, it is possible to use a dynamometer press having the reference Statif Manuel Imada SV-2 from the supplier Someco, and fitted with an Imada DPS-20 dynamometer from the same supplier.

The operator places a second medium 20 under the press, and in the example shown, this second medium is a standard sheet of photocopy paper, of A4 format, weighing 80 grams per square meter (g/m²), and white in color.

The second medium 20 has a first box 18 carrying an identifier 19, e.g. the number 1, 2, 3, or 4 of the test piece 15 in this example. The box 18 has a width of 2 centimeters (cm), for example.

A second box 21 adjacent to the first box 18 has a width of 3 cm, for example.

The operator measures the color L_{S2}*a_{S2}*b_{S2}* of the bare second medium 20.

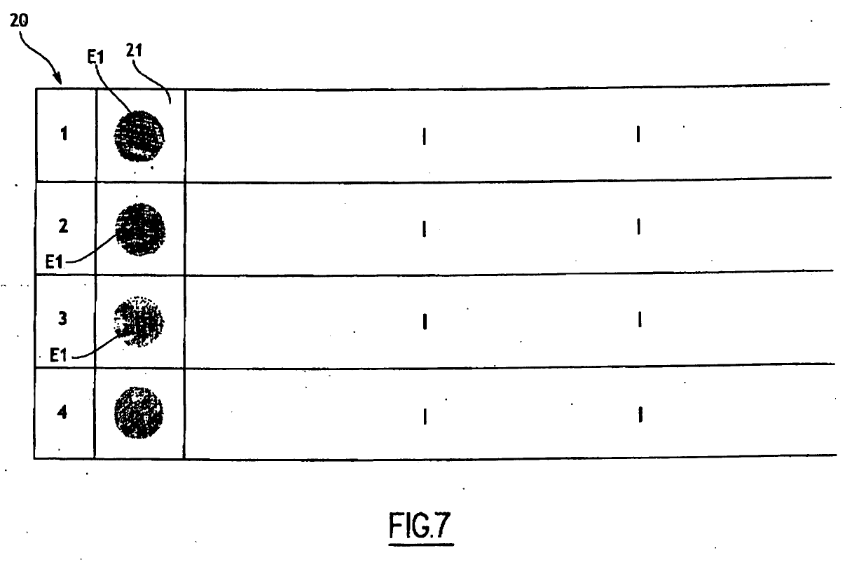

In the second box 21 of the second medium 20, the operator presses the test piece 15 statically against the second medium 20 so as to form a first imprint E1, as can be seen in FIG. 7. The pressure exerted on the test piece to make the imprint E1 is about 1 kilogram per square centimeter (kg/cm²), and it is exerted for a duration of 30 seconds (s) in the example described, at ambient temperature, i.e. about 24° C.+2° C. This corresponds to a reading of about 2.5 kg on the dynamometer.

In order to be able subsequently to evaluate transfer of the cosmetic, the operator measures the color L_{E1}*a_{E1}*b_{E1}* of the first imprint E1 on the second medium 20.

A total transfer reference is also measured by applying the composition directly to the second medium 20 or onto an identical medium, e.g. another standard sheet of photocopy paper weighing 80 g/m², white in color, and of A4 format from the same manufacturer, at ambient temperature, with the composition being smoothed by using a paint brush in order to obtain a deposit that is about 15 μm thick. It is allowed to try for about 30 min at ambient temperature.

After drying, the color L_{T}*a_{T}*b_{T}* of the deposit on the medium is measured, which corresponds to the color of a total transfer reference.

All of the color measurements can be performed using a colorimeter, e.g. of the CR 300 type sold by the supplier Minolta, after it has been calibrated, where the values L*, a*, and b* are the chromaticity coordinates in the 1976 CIE L*a*b* space.

In general, color difference between X and Y is defined by the following formula:
ΔE(X/Y)=\sqrt{(L_{X}*−L_{Y}*)²+(a_{X}*−a_{Y}*)²+(b_{X}*−b_{Y}*)²}
Transfer in percentage terms is defined by:

$$100 \times \frac{Δ E ( E1 / S2 )}{Δ E ( T / S2 )} , \\ i . e . \text{:} \frac{\sqrt{{( {L}_{E1}^{*} - {L}_{S2}^{*} )}^{2} + {( {a}_{E1}^{*} - {a}_{S2}^{*} )}^{2} + {( {b}_{E1}^{*} - {b}_{S2}^{*} )}^{2}}}{\sqrt{{( {L}_{T}^{*} - {L}_{S2}^{*} )}^{2} + {( {a}_{T}^{*} - {a}_{S2}^{*} )}^{2} + {( {b}_{T}^{*} - {b}_{S2}^{*} )}^{2}}}$$

Transfer of close to 100% means that the color of the imprint E1 is as intense as that of the total transfer reference.

The same operator can implement the method with all of the test pieces 15 for the same cosmetic, thereby providing a plurality of transfer values. In a variant, different operators can share the test pieces.

The second medium 20 with the various imprints E1 of the tested test pieces is shown in FIG. 7.

It is possible to calculate the mean of the transfer values obtained over a plurality of tests:
transfer mean:

$$\overline{tansfer} = \frac{1}{N} {\sum }_{i} {transfer}_{i}$$
standard deviation:

$${σ}_{transfer} = \sqrt{\frac{N {\sum }_{i} {transfer}_{i}^{2} - {( {\sum }_{i} {transfer}_{i} )}^{2}}{N ( N - 1 )}}$$
95% confidence range:

$$transfer \pm 1.96 \sqrt{\frac{{σ}_{transfer}}{N}}$$

where N designates the number of measurements.
Retention Test

The retention test is preferably performed using a first medium 1′ which differs from the first medium 1 described above.

The first medium 1′ shown in FIG. 8 is rectangular in shape having dimensions of 40 mm×70 mm, comprising a layer 2 of white-colored foam, e.g. a polyethylene foam having density of 33 kg/m³, thickness of 3 mm, e.g. a foam known under the trade reference RE40X70 EP3 and sold by the supplier Joint Technique Lyonnais Ind.

The first medium 1′ also comprises a second layer constituted by a covering 3, in particular an optionally occlusive covering made of flexible and adhesive plastics material, presenting a quality in use such that the application of a lipstick to this covering is similar to that which is obtained on lips.

The second layer constituted by the covering 3 is fixed, in particular by adhesive, onto the foam layer 2.

By way of example the covering 3 may be the transparent adhesive tape sold by the supplier 3M® under the trade reference Blenderm® FH5000-55113.

The first medium 1′ also has an adhesive surface 4 on its face opposite from its face for receiving the covering 3.

A protective film 5 covers the adhesive surface 4. This protective film 5 includes a pull tab 5a to make it easier to remove.

An implementation of the method of the invention for evaluating retention is described below with reference to FIGS. 9 to 14.

The operator measures the color L_{S2}*a_{S2}*b_{S2}* of the first face 6 of the first medium 1′ while it is still bare.

The operator then places the first medium 1′ on a hot-plate set to the temperature of 40° C., e.g. a hot-plate of the N 81076 type sold by the supplier Ficher Bioblock, and waits for the first face 6 of the first medium 1′ to reach a temperature of 33° C.±1° C., where the “first” face is defined as the face of the second layer 3 facing away from the adhesive surface 4. To measure the surface temperature of the first medium 1′, the operator can use an infrared thermometer.

Once the first medium 1′ is at the desired temperature, the operator applies a film F of cosmetic to a thickness of about 15 μm on the first face 6 of the first medium 1′, i.e. the face that is remote from the face that receives the adhesive surface 4.

The cosmetic, which is a lipstick for example is previously stored at 24° C.±2° C.

By way of example, the operation performed by the operator to deposit the film of lipstick may consist in drawing zigzags on the first medium 1′ in such a manner as to cover substantially all of its surface, while generally passing over any one point only once. The cosmetic is preferably applied to the first medium in a manner that is as representative as possible of the real conditions of application of the cosmetic.

An advantage of using a covering 3 of the Blenderm® type for receiving the deposit of a film of cosmetic, in particular of lipstick, is that it makes it possible to simulate applying a cosmetic onto the skin or the mucous membranes, for example, with the feel during application being similar. Furthermore, the behavior of the film of cosmetic on the covering is quite similar to that of the cosmetic on the skin or the mucous membranes.

Thereafter, the operator can smooth the deposited film with a paint brush.

The film is then allowed to dry while the first medium 1′ remains on the hot-plate, so that it remains at 33° C.±1° C. for a duration of 10 min in the present example.

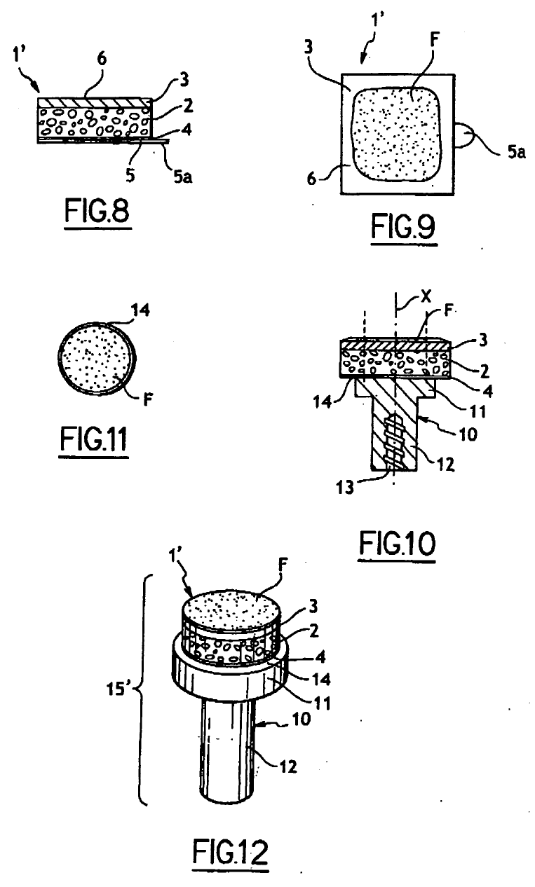

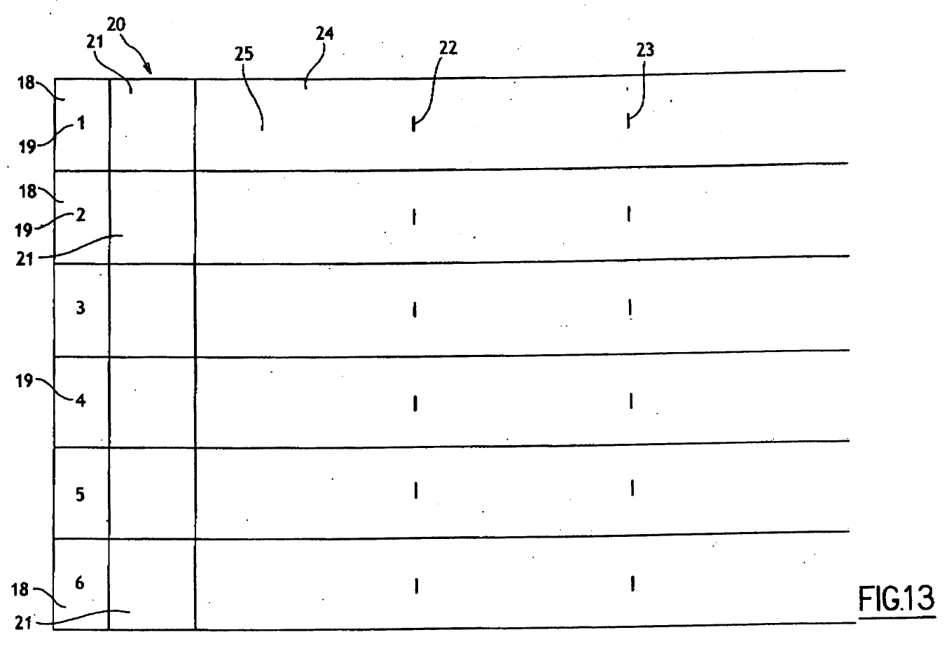

Thereafter, the operator proceeds to fix, in particular by using adhesive, the first medium 1′ on a sample carrier 10 identical to that used during the non-transfer test described above, and shown again in FIG. 10.

To do this, the operator removes the protective film 5 from the first medium 1′ and fixes the medium via its adhesive surface 4 on the sample carrier 10.

The first medium 1′ is stuck to the top face 14 of the head 11 in a zone where film F of cosmetic is uniform.

Using a punch, the operator cuts out from the first medium 1′ a circle of diameter that is slightly smaller than that of the head 11, e.g. equal to 18 mm.

The assembly comprising the sample carrier and the sample forms the test piece 15′ shown in FIG. 12.

A plurality of test pieces 15 can be prepared, e.g. six.

The color L_{F}*a_{F}*b_{F}* of the film F of cosmetic on each test piece 15′ is measured.

Thereafter, each sample carrier 12 is mounted in turn on a dynamometer press using the tapped hole 13 for screwing it to the press.

By way of example, it is possible to use the same dynamometer press as in the non-transfer test.

The operator places a second medium 20 under the press, and in the example shown in FIG. 13, the second medium is a sheet of standard photocopy paper, weighing 80 g/m², and white in color.

For each test piece, the second medium 20 comprises a first box 18 containing the identifier 19, e.g. the number of the test piece 15′, in particular a number in the range 1 to 6 in the present example. The box 18 is 2 cm wide.

A second box 21 adjacent to the first box 18 is 3 cm wide.

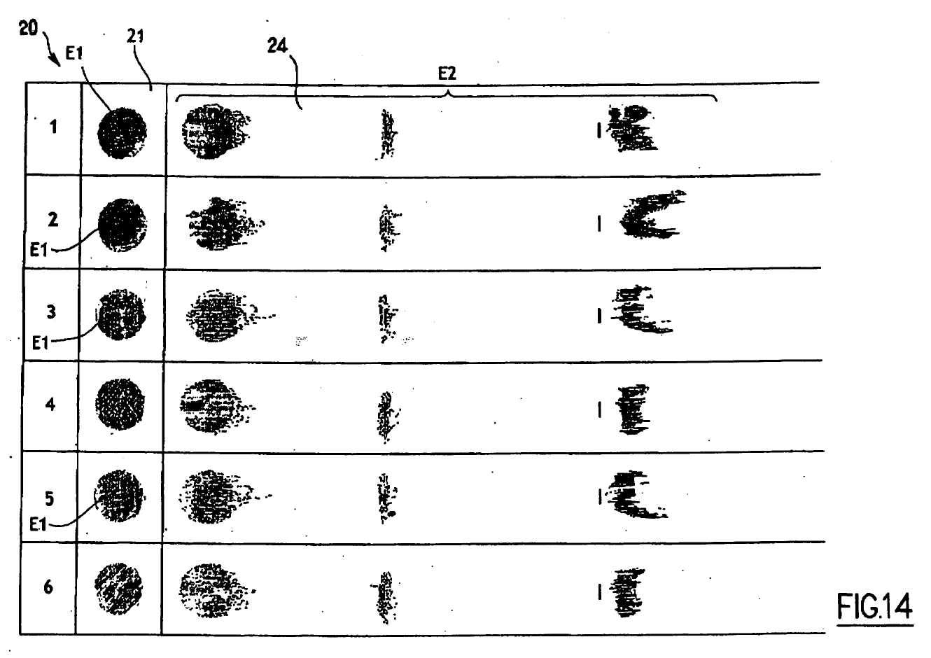

The operator presses the test piece 15′ in static manner against the second medium 20 in its second box 21 so as to transfer a first imprint E1 as can be seen in FIG. 14. The pressure exerted on the test piece to make the imprint E1 is about 300 g/cm², i.e. a reading of about 760 g on the dynamometer, and this pressure is applied for a duration of 30 s in the example shown, at ambient temperature, i.e. about 24° C.±2° C.

In a strip 24 adjacent to the box, the second medium 20 has a first mark 22 and a second mark 23 situated respectively at distances of 8 cm and 16 cm from the boundary between the strip-shaped box 24 on the second medium 20 and the second box 21.

The operator begins by depositing 20 microliters (μL) of water on the first mark 22 and 10 μL of oil on the second mark 23.

The oil deposited on the second mark 23 may be of food type, e.g. sunflower oil, or the oil sold under the trade mark ISIO 4®.

The operator then makes a second imprint E2 by pressing the test piece 15′ against the region 25 of the strip 24 adjacent to the box 21 with pressure of about 300 g/cm² and moving the second medium 20 relative to the test piece 15′ in rectilinear manner as soon as contact is achieved and at a speed of 1 centimeter per second (cm/s) over substantially the entire length of the strip 24, i.e. about 24 cm, so that the test piece 15′ passes through the marks 22 and 23.

The imprint E2 formed by the film of cosmetic as it passes in particular through the deposits of water and oil can be seen in FIG. 14. The imprint E2 is not subjected to color measurement but merely to qualitative observation.

At the end of this procedure, the operator measures the residual color L_{R}*a_{R}*b_{R}* of the film of cosmetic on the first medium 1′ in order to be able to determine the retention of the cosmetic.

Color measurements are performed using the same colorimeter as for the non-transfer test.

Retention is defined as a percentage by:

$$100 \times \frac{Δ E ( R / S1 )}{Δ E ( F / S1 )} , \\ i . e . \frac{\sqrt{{( {L}_{R}^{*} - {L}_{S1}^{*} )}^{2} + {( {a}_{R}^{*} - {a}_{S1}^{*} )}^{2} + {( {b}_{R}^{*} - {b}_{S1}^{*} )}^{2}}}{\sqrt{{( {L}_{F}^{*} - {L}_{S1}^{*} )}^{2} + {( {a}_{F}^{*} - {a}_{S1}^{*} )}^{2} + {( {b}_{F}^{*} - {b}_{S1}^{*} )}^{2}}}$$

Retention close to 100% means that the residual color of the film of cosmetic on the test piece remains very close to that of the same film as initially deposited.

The imprint E1 corresponding to different test pieces are made in the other locations available on the second medium 20 and numbered 2 to 6.

For a test with six test pieces, it is possible to use three operators, for example, each performing the test with two test pieces 15′.

Naturally, it will be possible to involve some other number of operators, in particular only one operator.

Finally, a second medium 20 is obtained as shown in FIG. 14, and it is possible to perform the following calculations:
mean retention:

$$\overline{retention} = \frac{1}{N} {\sum }_{i} {retention}_{i}$$
standard deviation:

$${σ}_{retention} = \sqrt{\frac{N {\sum }_{i} {retention}_{i}^{2} - {( {\sum }_{i} {retention}_{i} )}^{2}}{N ( N - 1 )}}$$
95% confidence range:

$$retention \pm 1.96 \sqrt{\frac{{σ}_{retention}}{N}}$$

where N designates the number of measurements.

Naturally, the invention is not limited to the implementations described above.

The methods described above may be modified without thereby going beyond the ambit of the present invention.

The pressure with which the test piece 15 or 15′ is pressed against the second medium 20 may lie in the range 200 g/cm² to 2 kg/cm², for example.

The time the film of cosmetic is allowed to dry after being deposited on the first medium may be modified and lie in the range 5 min to 1 hour, for example.

The diameter and the shape of the sample may also be varied.

The second medium need not be a sheet of paper, in particular it could comprise some other cellulose material.

In a variant, at least one of the first and second media may present a color that is other than white.

The second medium may have a single mark for receiving a single deposit of liquid or it may have more than two marks for receiving a corresponding number of deposits of liquids.

Some or all of the steps of the non-transfer and retention methods described above can be automated, in particular they can be performed by a robot instead of being performed by a human operator.

1. A method of in vitro evaluation of the transfer of a cosmetic comprising:
raising a first medium to a predetermined temperature;
depositing a film of the cosmetic on a first face of the first medium;
allowing the film to dry during a predetermined duration;
measuring the color (L_{S2}*a_{S2}*b_{S2}*) of a second medium and the color (L_{R}*a_{R}*b_{R}*) of a total transfer reference;
forming a first imprint of the film of cosmetic on the second medium by pressing the first face of the first medium in static manner against the second medium with predetermined pressure;
measuring the color (L_{E1}*a_{E1}*b_{E1}*) of the first imprint; and
evaluating the transfer of the cosmetic from the colors as measured in this way.

2. A method according to claim 1, wherein evaluating the transfer of the cosmetic comprises evaluating the ratio of the color difference between the first imprint and the second medium having no imprint, relative to the color difference between the total transfer reference and the second medium having no imprint.

3. A method according to claim 1, wherein the predetermined temperature to which the first medium is heated is of the order of 33° C.±1° C.

4. A method according to claim 1, comprising smoothing the deposited film of cosmetic.

5. A method according to claim 4, wherein smoothing the deposited film of cosmetic takes place prior to allowing it to dry.

6. A method according to claim 1, comprising fixing the first medium on a sample carrier.

7. A method according to claim 1, wherein the first medium comprises an adhesive surface on a second face opposite from the first face.

8. A method according to claim 1, comprising cutting out a sample for test purposes from the first medium, along an axis perpendicular to the plane of the first face of the first medium.

9. A method according to claim 6, comprising fixing the sample carrier on a dynamometer press.

10. A method according to claim 1, wherein the first medium is pressed in static manner against the second medium with pressure lying in the range 200 g/cm² to 2 kg/cm².

11. A method according to claim 1, wherein the first imprint of the film of cosmetic is formed on the second medium by pressing the first medium in static manner against the second medium with pressure of about 1 kg/cm².

12. A method according to claim 1, wherein the second medium is a sheet of white paper such as a standard photocopy sheet weighing 80 g/m².

13. A method according to claim 1, wherein the first and second media are white in color when they are free respectively of the film(s) of cosmetic and of the imprints.

14. A method according to claim 1, wherein the first medium comprises a layer of foam whose surface constitutes said first face.

15. A method according to claim 1, implemented at least in part by a robot.

16. A method according to claim 1, wherein the cosmetic is a lipstick.

17. A method of in vitro evaluation of the retention of a cosmetic, the method comprising:
raising a first medium to a predetermined temperature;
measuring the color (L_{S1}*a_{S1}*b_{S1}*) of a first face of the first medium;
depositing a film of the cosmetic on the first face of the first medium;
allowing the film of the cosmetic to dry during a first predetermined duration;
measuring the color (L_{F}*a_{F}*b_{F}*) of the film of cosmetic;
forming a first imprint of the film of cosmetic on a second medium by pressing the first face of the first medium in static manner against the second medium with predetermined pressure;
depositing at least one predetermined quantity of at least one liquid on the second medium at at least one predetermined location;
forming a second imprint of the film of cosmetic on the second medium by pressing the first face of the first medium against the second medium with predetermined pressure and by moving the second medium relative to the first face of the first medium at a predetermined speed and along a predetermined path passing through the deposit(s) of liquid;
measuring the residual color (L_{R}*a_{R}*b_{R}*) of the film of cosmetic on the first medium; and
evaluating the retention of the cosmetic on the basis of the colors as measured in this way.

18. A method according to claim 17, wherein evaluating the retention of the cosmetic comprises evaluating the ratio of the color difference between the film after the two imprints and the first face of the first medium having no film, relative to the color difference between the film before the first two imprints and the first face of the first medium having no film.

19. A method according to claim 17, wherein the predetermined temperature to which the first medium is heated is of the order of 33° C.±1° C.

20. A method according to claim 17, comprising smoothing the deposited film of cosmetic.

21. A method according to claim 17, wherein smoothing the deposited film of cosmetic takes place prior to allowing it to dry.

22. A method according to claim 17, comprising fixing the first medium on a sample carrier.

23. A method according to claim 17, wherein the first medium comprises an adhesive surface on a second face opposite from the first face.

24. A method according to claim 17, comprising cutting out a sample for test purposes from the first medium, along an axis perpendicular to the plane of the first face of the first medium.

25. A method according to claim 22, comprising fixing the sample carrier on a dynamometer press.

26. A method according to claim 17, wherein the first medium is pressed in static manner against the second medium with pressure lying in the range 200 g/cm² to 2 kg/cm².

27. A method according to claim 17, wherein the first imprint of the film of cosmetic is formed on the second medium by pressing the first medium in static manner against the second medium with pressure equal to about 300 g/cm².

28. A method according to claim 17, wherein the second medium is a sheet of white paper such as a standard photocopy sheet weighing 80 g/m².

29. A method according to claim 17, wherein the first and second media are white in color when they are free respectively of the film(s) of cosmetic and of the imprints.

30. A method according to claim 17, wherein the first medium comprises a layer of foam having a covering fixed thereto, the first face of the first medium that is to receive the film of cosmetic being a face of said covering.

31. A method according to claim 17, wherein the predetermined speed with which the second medium is moved relative to the first medium is about 1 cm/s.

32. A method according to claim 17, wherein the cosmetic is a lipstick.
